SEQUENCE LISTING (4 sequences)

SEQ ID NO: 1 - DNA, 2406 bp, Homo sapiens
  source 1..2406: mol_type = genomic DNA
agaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctatgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacgagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcccaaaaaaaaaa

SEQ ID NO: 2 - protein, 545 aa, Homo sapiens
MGRRGSAPGNGRTLRGCERGGRRRRSADSVMAEQVALSRTQVCGILREELFQGDAFHQSDTHIFIIMGASGDLAKKKIYPTIWWLFRDGLLPENTFIVGYARSRLTVADIRKQSEPFFKATPEEKLKLEDFFARNSYVAGQYDDAASYQRLNSHMNALHLGSQANRLFYLALPPTVYEAVTKNIHESCMSQIGWNRIIVEKPFGRDLQSSDRLSNHISSLFREDQIYRIDHYLGKEMVQNLMVLRFANRIFGPIWNRDNIACVILTFKEPFGTEGRGGYFDEFGIIRDVMQNHLLQMLCLVAMEKPASTNSDDVRDEKVKVLKCISEVQANNVVLGQYVGNPDGEGEATKGYLDDPTVPRGSTTATFAAVVLYVENERWDGVPFILRCGKALNERKAEVRLQFHDVAGDIFHQQCKRNELVIRVQPNEAVYTKMMTKKPGMFFNPEESELDLTYGNRYKNVKLPDAYERLILDVFCGSQMHFVRSDELREAWRIFTPLLHQIELEKPKPIPYIYGSRGPTEADELMKRVGFQYEGTYKWVNPHKL

SEQ ID NO: 3 - protein, 348 aa, Escherichia coli
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 4 - protein, 348 aa, Dickeya chrysanthemi
MERWFKSLFVLVLFFVFTASAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

(19) United States
(12) Patent Application Publication
(10) Pub. No.: US 2024/0024436 A1
(43) Pub. Date: Jan. 25, 2024

(54) METHOD FOR TREATING CANCER

(71) Applicant: CHILDREN'S MEDICAL CENTER CORPORATION, Boston, MA (US)

(72) Inventors: Yun-Cheol CHAE, Chestnut Hill, MA (US); Alejandro GUTIERREZ, Brookline, MA (US)

(73) Assignee: CHILDREN'S MEDICAL CENTER CORPORATION, Boston, MA (US)

(21) Appl. No.: 18/206,830

(22) Filed: Jun. 7, 2023

Related U.S. Application Data
(60) Provisional application No. 63/349,638, filed on Jun. 7, 2022.

(51) Int. Cl. A61K 38/50; A61P 35/00; C12N 15/113

(52) U.S. Cl. CPC A61K 38/50; A61P 35/00; C12N 15/1135; C12Y 305/01001

(57) ABSTRACT

Described herein are methods and compositions for treating cancer. Aspects of the invention relate to administering to a subject having cancer an asparaginase and an agent that inhibits G6PD. Another aspect of the invention relates to administering an asparaginase to a subject having cancer that comprises a G6PD deficiency.

### GOVERNMENT SUPPORT

This invention was made with Government support under Grant Nos. R01 CA249678 and CA193651 awarded by the National Institutes of Health. The Government has certain rights in the invention.

### FIELD OF THE INVENTION

The field of the invention relates to the treatment of cancer.

### SEQUENCE LISTING

The instant application contains a Sequence Listing which has been submitted electronically in XML format and is hereby incorporated by reference in its entirety. Said XML copy, created on Sep. 27, 2023, is named “701039-191760USPT_SL.xml” and is 8,114 bytes in size.

### CROSS REFERENCE TO RELATED APPLICATIONS

This application claims benefit under 35 U.S.C. § 119(e) of U.S. Provisional Application 63/349,638 filed on Jun. 7, 2022, the contents of which is incorporated herein by reference in its entirety.

### BACKGROUND

Colorectal cancer (CRC) is the second leading cause of cancer deaths worldwide, and its global incidence is rising. An estimated 96% of CRCs have mutations that activate canonical WNT/B-catenin signaling, and these mutations promote intestinal transformation. However, oncogenic B-catenin activity is difficult to inhibit directly, and targeting ligands that active WNT signaling, such as R-spondin lead to significant bone toxicity with pathologic fractures. See e.g., Hinze et al., Cancer Discov. 10, 1690-1705 (2020); Yaeger et al., Cancer Cell 33, 125-136 (2018); Nusse et al., Cell 169, 985-999 (2017); Tan et al., Annals of Oncology (2018); the contents of each of which are incorporated herein by reference in their entireties.

Leukemia is the most common of pediatric cancers accounting for about 30% of diagnoses. There are two main subtypes; acute lymphoblastic leukemia (ALL) and acute myeloid leukemia (AML). AML is less common, accounting for approximately 18% of childhood leukemia diagnoses. These leukemia types also occur in adults, and AML becomes more common in older individuals. The etiology of the two subtypes is likely quite different based on both cell lineage and epidemiological studies of incidence and risk factors. Aggressive chemotherapies are required to improve the prognosis of patients diagnoses with leukemias such as ALL or AML.

Asparaginase, a bacterial enzyme that depletes the nonessential amino acid asparagine, is an integral component of acute leukemia therapy. However, despite its efficacy in ALL, asparaginase has been used only occasionally in treatment of other cancers. In colorectal cancers, for instance, 80-85% of colorectal cancer patients have an APC mutation, and these tumors are resistant to asparaginase. Therefore, there is a great need for better understanding mechanisms of asparaginase resistance as well as methods that can be applied in tumors to induce asparaginase activity.

### SUMMARY OF THE INVENTION

The invention described herein is related, in part, to the discovery that inhibition of G6PD sensitized cancer cells, e.g., colorectal cancer cells, to asparaginase. Accordingly, one aspect of the invention described herein provides a method for treating cancer comprising administering to a subject having cancer an asparaginase and an agent that inhibits G6PD.

In one embodiment of any aspect described herein, the cancer is selected from the list consisting of a carcinoma, a melanoma, a sarcoma, a myeloma, a leukemia, and a lymphoma.

In one embodiment of any aspect described herein, the cancer is a solid tumor.

In another embodiment of any aspect described herein, the cancer is a colon cancer.

In another embodiment of any aspect described herein, the leukemia is acute myeloid leukemia (AML), Chronic myeloid leukemia (CML), Acute lymphocytic leukemia (ALL), and Chronic lymphocytic leukemia (CLL).

In another embodiment of any aspect described herein, the cancer is resistant to an asparaginase.

In another embodiment of any aspect described herein, the cancer is not resistant to an asparaginase.

In another embodiment of any aspect described herein, the asparaginase is selected from the group consisting of: L-asparaginase (Elspar), pegaspargase (PEG-asparaginase; Oncaspar), SC-PEG asparaginase, Calaspargase pegol (Cal-PEG; SHP663), Erwinia asparaginase (Erwinaze), cristantaspase, and Asparaginase medac.

In another embodiment of any aspect described herein, the agent that inhibits G6PD is selected from the group consisting of a small molecule, an antibody, a peptide, a genome editing system, an antisense oligonucleotide, and an RNAi.

In another embodiment of any aspect described herein, the RNAi is a microRNA, an siRNA, or a shRNA.

In another embodiment of any aspect described herein, inhibiting G6PD is inhibiting the expression level and/or activity of G6PD.

In another embodiment of any aspect described herein, the expression level and/or activity of G6PD is inhibited by at least 50%, at least 60%, at least 70%, at least 80%, at least 90%, or more as compared to an appropriate control.

In another embodiment of any aspect described herein, the cancer is metastatic.

In another aspect, described herein is a method for treating cancer, the method comprising: (a) obtaining a biological sample from a subject having cancer; (b) assaying the sample and identifying a deficiency in G6PD; and (c) administering an asparaginase to a subject who has been identified with G6PD deficiency.

In another aspect, described herein is a method of treating cancer, the method comprising: (a) receiving the results of an assay that identifies a subject as having a deficiency in G6PD; and (b) administering an asparaginase to a subject who has been identified as having a deficiency in G6PD.

In one embodiment of any aspect described herein, the biological sample is a biopsied sample, a tissue sample, or a blood sample.

In another embodiment of any aspect described herein, the biopsied sample is a tumor sample.

In another embodiment of any aspect described herein, the subject has previously been administered an anti-cancer therapy.

In another embodiment of any aspect described herein, the subject has not previously been administered an anti-cancer therapy.

### Definitions

For convenience, the meaning of some terms and phrases used in the specification, examples, and appended claims, are provided below. Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with cancer, e.g., leukemia, or colon cancer. The term “treating” includes reducing or alleviating at least one adverse effect or symptom of cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic (e.g., an agent that inhibits G6PD and/or asparaginase) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a disease or disorder in need of treatment (e.g., cancer) or one or more complications related to such a disease or disorder, and optionally, have already undergone treatment for the disease or disorder or the one or more complications related to the disease or disorder. Alternatively, a subject can also be one who has not been previously diagnosed as having such disease or disorder or related complications. For example, a subject can be one who exhibits one or more risk factors for the disease or disorder or one or more complications related to the disease or disorder or a subject who does not exhibit risk factors.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit G6PD, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity, e.g. activity and specificity of a native or reference polypeptide (e.g., XCL1) is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity. A functional fragment can comprise conservative substitutions of the sequences disclosed herein.

In some embodiments, the polypeptide described herein can be a variant of a polypeptide sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a protein or fragment thereof that retains activity of the native or reference polypeptide. A wide variety of, for example, PCR-based, site-specific mutagenesis approaches are known in the art and can be applied by the ordinarily skilled artisan to generate and test artificial variants.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelrys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

As used herein, the term “cancer therapy” or “cancer treatment” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

The terms “increased”, “increase”, “enhance”, “activate” are all used herein to refer to an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The term “improve”, when applied to a score in a standardized scale or rating, e.g., for disease symptoms or severity, means a favorable change in the scale or rating on that scale.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to a reference level, for example, a decrease of at least about 20%, or at least about 30%, or at least about 40%, or at least about 50%, or at least about 60%, or at least about 70%, or at least about 80%, or at least about 90%, or up to and including a 100% decrease. As used herein, “reduction” or “inhibition” does not encompass a complete inhibition or reduction as compared to a reference level. “Complete inhibition” is a 100% inhibition as compared to an appropriate control.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with cancer, or a biological sample that has not been contacted with an agent disclosed herein).

As used herein, an “appropriate control” refers to an untreated, otherwise identical cell or population (e.g., a subject who was not administered an agent described herein, or was administered by only a subset of agents described herein, as compared to a non-control cell).

The term “modulation,” when applied to target gene expression refers to stimulation; i.e., a decrease in expression. This modulation can be measured in ways which are routine in the art, for example by Northern blot assay, RNAse protection assay or reverse transcriptase PCR for measurement of transcription or splicing products or mRNA, or by Western blot, ELISA or immunoprecipitation assay of protein expression. This modulation can also be measured using assays of enzymatic activity, e.g., G6PD enzymatic activity, or by measuring abundance of the substrates and products of enzymatic reactions, e.g., NADP and NADPH.

The term “therapeutically effective amount” refers to an amount of an G6PD inhibitor or pharmaceutical composition comprising a G6PD inhibitor as described herein that is sufficient to provide a particular effect when administered to a typical subject. An effective amount as used herein would also include an amount sufficient to delay the development of a symptom of a disease, alter the course of a symptom of a disease (for example but not limited to, slow the progression of a symptom of a disease), or reverse a symptom of a disease. Thus, it is not possible to specify an exact “effective amount” for every situation, however, for any given case, an appropriate “effective amount” can be determined by one of ordinary skill in the art using only routine experimentation.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1a-1b show a CRISPR screen for modifiers of asparaginase response in APC-mutant colorectal cancer. FIG. 1a Cas9-expressing HCT-15 cells were transduced with the Brunello guide RNA library in biologic duplicates. Each group was split into treatment with vehicle or asparaginase, and guide RNA representation was assessed after 10 days of treatment. FIG. 1b Genes covered by the Brunello library are shown ranked by significance of depletion in asparaginase-treated conditions, as assessed by negative selection score.

FIGS. 2a-2g show G6PD shRNA depletion induces asparaginase sensitivity in colorectal cancer cell lines. FIG. 2a Colorectal cancer cell lines, HCT-15 and HT-29, or CCD841 cells derived from normal human intestinal epithelium, were transduced with the indicated shRNAs, and knockdown efficiency was assessed by RT-PCR analysis for expression of the indicated gene, normalized to β-actin. Significance was calculated using one-way ANOVA with Dunnett's adjustment for multiple comparisons. FIG. 2b HCT-15 cells were transduced with the indicated shRNAs, G6PD activity assay was assessed. FIGS. 2c-2g Colorectal cancer cell lines, HCT-15 (FIG. 2c-2d), HCT-29 (FIG. 2e), or CCD841 (FIG. 2f-2g) cells derived from normal human intestinal epithelium, were transduced with the indicated shRNAs, and treated with the indicated doses of asparaginase. Relative viability was assessed by counting viable cells based on trypan blue vital dye staining, with cell counts normalized to those in shLuc-transduced, no-asparaginase controls. FIG. 2d Western blot analysis of G6PD protein expression levels in HCT-15 cells after treatment with indicated shRNAs.

FIGS. 3a-3c show small molecule G6PD inhibition induces asparaginase sensitivity CRC cell lines. FIG. 3a colorectal cell lines, HCT-15 and HT-29 were treated with small molecule inhibitor G6PDi at the indicated concentrations and activity of G6PD was assessed. FIGS. 3b-3c Colorectal cancer cell lines, HCT-15 (FIG. 3b) and HCT-29 (FIG. 3c) were treated with vehicle or G6PDi-1 (30 μM) and with the indicated doses of asparaginase. Relative viability was assessed by counting viable cells based on trypan blue vital dye staining, with cell counts normalized to those in cells with no-asparaginase controls.

FIGS. 4a-4b show small molecule inhibition induces asparaginase sensitivity in organoid models of Apc;Kras;Tp53 CRC. FIG. 4a Apc-deficient organoids with mutations of Tp53 and Kras were cultured in basal medium and treated with vehicle, asparaginase (500 U/L), G6PDi-1 (100 μM), or combo (500 U/L asparaginase+100 μM G6PDi-1). FIG. 4b Viability of organoids from FIG. 4a was assessed by counting viable organoids. Data is relative to vehicle treatment group.

FIGS. 5a-5b show that G6PD knock-out induces asparaginase sensitivity in CRC cell line. FIG. 5a, Western blot analysis of G6PD protein expression levels in parental HCT-15 cells (G6PD wild-type) and two distinct G6PD knock-out (KO) clones generated by CRISPR-Cas9 induced gene deletion. FIG. 5b, control (G6PD wild-type) or G6PD knockout (KO) clones of the HCT-15 colorectal cancer cell line were treated with asparaginase at the indicated doses, and relative cell viability was assessed by counting the number of live cells based on trypan blue vital dye staining, with cell counts normalized to those in cells with no-asparaginase controls.

FIG. 6 shows that reactive oxygen species levels in G6PD deficient cells are not further elevated by asparaginase treatment. HCT-15 cells were transduced with negative control shRNA (shLuciferase, shLuc) or with two distinct G6PD-targeting shRNAs (shG6PD #1 and #2), treated with asparaginase at the indicated doses, and reactive oxygen species (ROS) levels were assessed using the Cellular ROS Assay Kit (Abcam, product number ab113851) in shLuc (control). ROS levels were measured by fluorescence microplate reader, and were normalized to those in shLuc-transduced, no-asparaginase controls. These findings show that ROS production is not the major mechanism through which G6PD deficiency induces asparaginase sensitivity. Results are shown as means±SEM; n=3. ****P<0.0001.

FIG. 7 shows that inhibiting the nonoxidative branch of the pentose phosphate pathway, which requires the RPIA and RPE enzymes, does not mediate asparaginase sensitivity. HCT-15 colorectal cancer cells were transduced with a so-called CRISPR interference system, consisting of a dCas9-KRAB expression construct and guide RNAs targeting the promoters of RPIA and RPE, to inhibit expression of these enzymes. Cells were then treated with vehicle or asparaginase at the indicated doses. Relative viability was assessed by counting viable cells based on trypan blue vital dye staining, with cell counts normalized to those in cells with no-asparaginase controls. None of the differences were statistically significant at the p<0.05 significance level.

FIGS. 8A-8B show that asparaginase treatment induces a further increase of NADP/NADPH ratio in G6PD depleted cells. FIG. 8a, HCT-15 cells were transduced with the indicated shRNAs and with the indicated doses of asparaginase, and the ratio of NADP to NADPH was measured using the NADP/NADPH Assay Kit (abcam product #ab65349). Relative NADP/NADPH ratio was assessed by microplate reader based on colorimetric signals (OD 450 nm), with colorimetric signal normalized to those in shLuc cells with no-asparaginase controls. FIG. 8b, the colorectal cancer cell line HCT-15 was treated with vehicle, asparaginase (500 U/L), G6PDi-1 (30 μM) or combination treatment. ROS level was measured by NADP/NADPH Assay Kit (ab65349) in indicated cells. Relative NADP/NADPH ratio was assessed by microplate reader based on colorimetric signals (OD 450 nm), with colorimetric signal normalized to those in shLuc cells with no-asparaginase controls.

FIG. 9 shows Asparaginase treatment induces further increase of NADP/NADPH ratio in G6PD knock-out cells. HCT-15 cells that were either G6PD wild-type (WT) parental cells, or G6PD knock-out clones #1 and #2, were treated by vehicle or asparaginase (500 U/L). NADP/NADPH ratio was measured by liquid chromatography/mass spectrometry, and levels are shown normalized to those in HCT-15 cells with no-asparaginase controls. Note that the data in FIGS. 8A, 8B and 9 show that asparaginase treatment further increases the ratio of NADP/NADPH in G6PD-deficient cells, arguing that it exacerbates NAPDH deficiency caused by loss of G6PD

FIG. 10 shows generation of G6pdx deficient mouse intestinal organoid models of colorectal cancer. Left panel, Mouse intestinal organoids with Apc deficiency, Kras activating mutations, and Tp53 loss (so-called AKP organoids) were transduced with two distinct control shRNAs (shGFP and shLuciferase), or two distinct shRNAs targeting G6pdx (the mouse G6PD gene). Knockdown of G6pdx protein expression was confirmed by Western blot analysis. Right panel, experimental design for the experiment shown in FIG. 11.

FIG. 11 shows that G6pdx deficient mouse intestinal organoid models of colorectal cancer are asparaginase sensitive. Organoids were transduced with control (shLuc) and two distinct shG6pdx-targeting shRNAs (see FIG. 10), and injected subcutaneously into immunodeficient mice. After engraftment of tumors (defined as tumors reaching a volume of 150 mm3), mice were treated with vehicle (phosphate-buffered saline, PBS) or asparaginase (1,000 U/kg), and tumor volumes were followed. Asparaginase or PBS were re-dosed at the time points indicated by the black arrows. Note that asparaginase has significant therapeutic activity against G6pdx-deficient, but not control, organoid models of colorectal cancer.

### DETAILED DESCRIPTION

The invention described herein is related, in part, to the discovery that inhibition of G6PD via the administration of a G6PD inhibitor sensitized cancer cells, e.g., colorectal cells, to treatment with asparaginase. Specifically, treatment with a G6PD inhibitor sensitized asparaginase-resistant cells to treatment with asparaginase. Accordingly, one aspect of the invention is a method for treating cancer comprising administering to a subject having cancer an asparaginase and an agent that inhibits G6PD.

Methods and compositions described herein require that the levels and/or activity of G6PD are inhibited. As used herein, “Glucose-6-phospate dehydrogenase (G6PD)” refers to a cytosolic enzyme that is responsible for the first step in the pentose phosphate pathway, a series of chemical reactions that convert glucose to ribose-5-phospate. G6PD's main function is to produce NADPH, a key electron donor in the defense against oxidizing agents and in reductive biosynthetic reactions. G6PD sequences are known for a number of species, e.g., human G6PD (NCBI Gene ID: 2539) mRNA (e.g., isoform a: NCBI Ref Seq NM_000402.4, SEQ ID NO: 1) and polypeptide (e.g., isoform a: NCBI Ref Seq NP_000393.4, SEQ ID NO: 2). G6PD can refer to human G6PD, including naturally occurring variants, molecules, and alleles thereof. G6PD refers to the mammalian G6PD, e.g., mouse, rat, rabbit, dog, cat, cow, horse, pig, and the like. The nucleic sequence of SEQ ID NO:1 comprises the nucleic sequence which encodes G6PD.

| SEQ ID NO: 1 is a nucleic acid sequence that encodes isoform a of | |
|---|---|
| human G6PD. | |
| 1 | agaggcaggg gctggcctgg gatgcgcgcg cacctgccct cgccccgccc cgcccgcacg |
| 61 | aggggtggtg gccgaggccc cgccccgcac gcctcgcctg aggcgggtcc gctcagccca |
| 121 | ggcgcccgcc cccgcccccg ccgattaaat gggccggcgg ggctcagccc ccggaaacgg |
| 181 | tcgtacactt cggggctgcg agcgcggagg gcgacgacga cgaagcgcag acagcgtcat |
| 241 | ggcagagcag gtggccctga gccggaccca ggtgtgcggg atcctgcggg aagagctttt |
| 301 | ccagggcgat gccttccatc agtcggatac acacatattc atcatcatgg gtgcatcggg |
| 361 | tgacctggcc aagaagaaga tctaccccac catctggtgg ctgttccggg atggccttct |
| 421 | gcccgaaaac accttcatcg tgggctatgc ccgttcccgc ctcacagtgg ctgacatccg |
| 481 | caaacagagt gagcccttct tcaaggccac cccagaggag aagctcaagc tggaggactt |
| 541 | ctttgcccgc aactcctatg tggctggcca gtacgatgat gcagcctcct accagcgcct |
| 601 | caacagccac atgaatgccc tccacctggg gtcacaggcc aaccgcctct tctacctggc |
| 661 | cttgcccccg accgtctacg aggccgtcac caagaacatt cacgagtcct gcatgagcca |
| 721 | gataggctgg aaccgcatca tcgtggagaa gcccttcggg agggacctgc agagctctga |
| 781 | ccggctgtcc aaccacatct cctccctgtt ccgtgaggac cagatctacc gcatcgacca |
| 841 | ctacctgggc aaggagatgg tgcagaacct catggtgctg agatttgcca acaggatctt |
| 901 | cggccccatc tggaaccggg acaacatcgc ctgcgttatc ctcaccttca aggagccctt |
| 961 | tggcactgag ggtcgcgggg gctatttcga tgaatttggg atcatccggg acgtgatgca |
| 1021 | gaaccaccta ctgcagatgc tgtgtctggt ggccatggag aagcccgcct ccaccaactc |
| 1081 | agatgacgtc cgtgatgaga aggtcaaggt gttgaaatgc atctcagagg tgcaggccaa |
| 1141 | caatgtggtc ctgggccagt acgtggggaa ccccgatgga gagggcgagg ccaccaaagg |
| 1201 | gtacctggac gaccccacgg tgccccgcgg gtccaccacc gccacttttg cagccgtcgt |
| 1261 | cctctatgtg gagaatgaga ggtgggatgg ggtgcccttc atcctgcgct gcggcaaggc |
| 1321 | cctgaacgag cgcaaggccg aggtgaggct gcagttccat gatgtggccg gcgacatctt |
| 1381 | ccaccagcag tgcaagcgca acgagctggt gatccgcgtg cagcccaacg aggccgtgta |
| 1441 | caccaagatg atgaccaaga agccgggcat gttcttcaac cccgaggagt cggagctgga |
| 1501 | cctgacctac ggcaacagat acaagaacgt gaagctccct gacgcctatg agcgcctcat |
| 1561 | cctggacgtc ttctgcggga gccagatgca cttcgtgcgc agcgacgagc tccgtgaggc |
| 1621 | ctggcgtatt ttcaccccac tgctgcacca gattgagctg gagaagccca agcccatccc |
| 1681 | ctatatttat ggcagccgag gccccacgga ggcagacgag ctgatgaaga gagtgggttt |
| 1741 | ccagtatgag ggcacctaca agtgggtgaa cccccacaag ctctgagccc tgggcaccca |
| 1801 | cctccacccc cgccacggcc accctccttc ccgccgcccg accccgagtc gggaggactc |
| 1861 | cgggaccatt gacctcagct gcacattcct ggccccgggc tctggccacc ctggcccgcc |
| 1921 | cctcgctgct gctactaccc gagcccagct acattcctca gctgccaagc actcgagacc |
| 1981 | atcctggccc ctccagaccc tgcctgagcc caggagctga gtcacctcct ccactcactc |
| 2041 | cagcccaaca gaaggaagga ggagggcgcc cattcgtctg tcccagagct tattggccac |
| 2101 | tgggtctcac tcctgagtgg ggccagggtg ggagggaggg acgaggggga ggaaaggggc |
| 2161 | gagcacccac gtgagagaat ctgcctgtgg ccttgcccgc cagcctcagt gccacttgac |
| 2221 | attccttgtc accagcaaca tctcgagccc cctggatgtc ccctgtccca ccaactctgc |
| 2281 | actccatggc caccccgtgc cacccgtagg cagcctctct gctataagaa aagcagacgc |
| 2341 | agcagctggg acccctccca acctcaatgc cctgccatta aatccgcaaa cagcccaaaa |
| 2401 | aaaaaa |
| SEQ ID NO: 2 is an amino acid sequence of Isoform 1 of human G6PD. | |
| 1 | mgrrgsapgn grtlrgcerg grrrrsadsv maeqvalsrt qvcgilreel fqgdafhqsd |
| 61 | thifiimgas gdlakkkiyp tiwwlfrdgl lpentfivgy arsrltvadi rkqsepffka |
| 121 | tpeeklkled ffarnsyvag qyddaasyqr lnshmnalhl gsqanrlfyl alpptvyeav |
| 181 | tknihescms qigwnriive kpfgrdlqss drlsnhissl fredqiyrid hylgkemvqn |
| 241 | lmvlrfanri fgpiwnrdni acviltfkep fgtegrggyf defgiirdvm qnhllqmlcl |
| 301 | vamekpastn sddvrdekvk vlkcisevqa nnvvlgqyvg npdgegeatk gylddptvpr |
| 361 | gsttatfaav vlyvenerwd gvpfilrcgk alnerkaevr lqfhdvagdi fhqqckrnel |
| 421 | virvqpneav ytkmmtkkpg mffnpeesel dltygnrykn vklpdayerl ildvfcgsqm |
| 481 | hfvrsdelre awriftpllh qielekpkpi pyiygsrgpt eadelmkrvg fqyegtykwv |
| 541 | nphkl |

Another aspect of the invention is a method for treating cancer comprising administering to a subject having cancer asparaginase, wherein the cancer comprises a mutation that results in G6PD deficiency. In one embodiment, prior to administration, a subject is identified with having cancer comprising a G6PD deficiency. G6PD deficiency is one of the most common inherited enzyme defects and is widespread in human populations from tropical and subtropical areas such as Sub-Saharan Africa, and in the Mediterranean area, as well as individuals whose ancestry traces to those populations.

G6PD deficiency can be identified in a biological sample obtained from the subject, for example, via genomic sequencing of the biological sample and comparing the sequence to a wild-type sequence (i.e., a sequence not having a mutation). Alternatively, G6PD deficiency can be identified using enzymatic assays that measure activity of this enzyme in a biological sample. Exemplary biological samples include a tissue sample or a blood sample. A biological sample can be obtained from a subject using standard techniques known in the art. For example, a biological sample can be obtained from a biopsy, or a standard blood draw.

Another aspect of the invention described herein provides a method of treating cancer comprising: (a) obtaining a biological sample from a subject, (b) identifying G6PD deficiency, and (c) administering asparaginase to a subject who has been identified as having a G6PD deficiency.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

In one embodiment, the cancer is a carcinoma, a melanoma, a sarcoma, a myeloma, a leukemia, and a lymphoma.

A carcinoma is a cancer that originates in an epithelial tissue. Carcinomas account for approximately 80-90% of all cancers. Carcinomas can affect organs or glands capable of secretion (e.g., breasts, lung, prostate, colon, or bladder). There are two subtypes of carcinomas: adenocarcinoma, which develops in an organ or gland, and squamous cell carcinoma, which originates in the squamous epithelium. Adenocarcinomas generally occur in mucus membranes, and are observed as a thickened plaque-like white mucosa. They often spread easily through the soft tissue where they occur. Exemplary adenocarcinomas include, but are not limited to, lung cancer, prostate cancer, pancreatic cancer, esophageal cancer, and colorectal cancer. Squamous cell carcinomas can originate from any region of the body. Examples of carcinomas include, but are not limited to, prostate cancer, colorectal cancer, microsatellite stable colon cancer, microsatellite instable colon cancer, hepatocellular carcinoma, breast cancer, lung cancer, small cell lung cancer, non-small cell lung cancer, lung adenocarcinoma, melanoma, basal cell carcinoma, squamous cell carcinoma, renal cell carcinoma, ductal carcinoma in situ, ductal carcinoma.

Sarcomas are cancers that originate in supportive and connective tissues, for example bones, tendons, cartilage, muscle, and fat. Sarcoma tumors usually resemble the tissue in which they grow. Non-limiting examples of sarcomas include, Osteosarcoma or osteogenic sarcoma (originating from bone), Chondrosarcoma (originating from cartilage), Leiomyosarcoma (originating from smooth muscle), Rhabdomyosarcoma (originating from skeletal muscle), Mesothelial sarcoma or mesothelioma (originate from membranous lining of body cavities), Fibrosarcoma (originating from fibrous tissue), Angiosarcoma or hemangioendothelioma (originating from blood vessels), Liposarcoma (originating from adipose tissue), Glioma or astrocytoma (originating from neurogenic connective tissue found in the brain), Myxosarcoma (originating from primitive embryonic connective tissue), or Mesenchymous or mixed mesodermal tumor (originating from mixed connective tissue types).

Melanoma is a type of cancer forming from pigment-containing melanocytes. Melanoma typically develops in the skin, but can occur in the mouth, intestine, or eye.

Myelomas are cancers that originate in plasma cells of bone marrow. Non-limiting examples of myelomas include multiple myeloma, plasmacytoma and amyloidosis.

Lymphomas develop in the glands or nodes of the lymphatic system (e.g., the spleen, tonsils, and thymus), which purifies bodily fluids and produces white blood cells, or lymphocytes. Unlike leukemia, lymphomas form solid tumors. Lymphoma can also occur in specific organs, for example the stomach, breast, or brain; this is referred to as extranodal lymphomas). Lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma. Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma.

Leukemias (also known as “blood cancers”) are cancers of the bone marrow, which is the site of blood cell production. Leukemia is often associated with the overproduction of immature white blood cells. Immature white blood cells do not function properly, rendering the patient prone to infection. Leukemia additionally affects red blood cells, and can cause poor blood clotting and fatigue due to anemia.

In one embodiment, the cancer is a colorectal cancer.

In one embodiment, the leukemia is acute myeloid leukemia (AML), Chronic myeloid leukemia (CML), Acute lymphocytic leukemia (ALL), and Chronic lymphocytic leukemia (CLL). Examples of leukemia include, but are not limited to, Myelogenous or granulocytic leukemia (malignancy of the myeloid and granulocytic white blood cell series), Lymphatic, lymphocytic, or lymphoblastic leukemia (malignancy of the lymphoid and lymphocytic blood cell series), and Polycythemia vera or erythremia (malignancy of various blood cell products, but with red cells predominating).

In one embodiment, the cancer is a solid tumor. Non-limiting examples of solid tumors include Adrenocortical Tumor, Alveolar Soft Part Sarcoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carinomas.

In one embodiment of any aspect, the cancer is resistant to a cancer therapy. In one embodiment of any aspect, the cancer is resistant to an asparaginase. A cancer resistant to a therapy, for example, asparaginase, is one that previously responded to the treatment but is now capable of growing or persisting despite the presence of continued treatment. Resistance to a therapy can occur due to, e.g., acquired mutations in the cancer cell, gene amplification in the cancer cell, or the cancer cell develops mechanisms to prevent the uptake of the treatment. In one embodiment of any aspect, the cancer is not resistant to a cancer therapy or asparaginase.

In one embodiment, the cancer is metastatic (e.g., the cancer has disseminated from its primary location to at least one secondary location).

In one embodiment, the cancer is resistant to a cancer therapy. In another embodiment, the cancer is not resistant to a cancer therapy.

In one embodiment, the cancer has relapsed following administration of a cancer therapy.

In one embodiment, the cancer therapy is selected from the group consisting of chemotherapy, radiation therapy, immunotherapy, surgery, hormone therapy, stem cell therapy, targeted therapy, gene therapy, and precision therapy.

In other embodiments of any method described herein, the cancer therapy is selected from the group consisting of growth inhibitory agents, cytotoxic agents, anti-angiogenesis agents, apoptotic agents, anti-tubulin agents, anti-HER-2 antibodies, anti-CD20 antibodies, an epidermal growth factor receptor (EGFR) antagonist, a HER1/EGFR inhibitor, a platelet derived growth factor inhibitor, a COX-2 inhibitor, an interferon, and a cytokine (e.g., G-CSF, granulocyte-colony stimulating factor).

In other embodiments, the cancer therapy is selected from the group consisting of 13-cis-retinoic acid, 2-CdA, 2-Chlorodeoxyadenosine, 5-Azacitidine, azacytidine, 5-Fluorouracil, 5-FU, 6-Mercaptopurine, 6-MP, 6-TG, 6-Thioguanine, abiraterone acetate, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Axitinib, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225, Cabazitaxel, Calcium Leucovorin, Campath® Camptosar® Camptothecin-11, Capecitabine, Caprelsa® Carac™ Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Crizotinib, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, Denosumab, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eculizumab, Efudex®, Eligard™, Ellence™, Eloxatin™, Elspar®, Emcyt®, Epirubicin, Epoetin Alpha, Erbitux, Eribulin, Erlotinib, Erwinia L-asparaginase, Estramustine, Ethyol, Etopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, Gefitinib, Gemcitabine, Gemtuzumab ozogamicin, Gemzar, Gleevec™, Gliadel® Wafer, Goserelin, Granulocyte-Colony Stimulating Factor (G-CSF), Granulocyte Macrophage Colony Stimulating Factor (GM-CSF), Halaven®, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Inlyta®, Interferon alpha, Interferon Alpha-2b (PEG Conjugate), Interleukin-2, Interleukin-11, Intron A® (interferon alpha-2b), Ipilimumab, Iressa®, Irinotecan, Isotretinoin, Ixabepilone, Ixempra™, Jevtana®, Kidrolase (t), Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, Octreotide, Octreotide acetate, Ofatumumab, Oncospar®, Oncovin®, Ontak®, Onxal™, Oprelvekin, Orapred®, Orasone®, Oxaliplatin, Paclitaxel, Paclitaxel Protein-bound, Pamidronate, Panitumumab, Panretin®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolia®, Prolifeprospan 20 with Carmustine Implant, Provenge®, Purinethol®, Raloxifene, Revlimid®, Rheumatrex®, Rituxan®, Rituximab, Roferon-A® (Interferon Alfa-2a), Romiplostim, Rubex®, Rubidomycin hydrochloride, Sandostatin®, Sandostatin LAR®, Sargramostim, Sipuleucel-T, Soliris®, Solu-Cortef®, Solu-Medrol®, Sorafenib, SPRYCEL™, STI-571, Streptozocin, SU11248, Sunitinib, Sutent®, Tamoxifen, Tarceva®, Targretin®, Tasigna®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, Valrubicin, Valstar, vandetanib, VCR, Vectibix™, Velban®, Velcade®, Vemurafenib, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, Xalkori capsules, Xeloda®, Xgeva®, Yervoy®, Zanosar®, Zelboraf, Zevalin™, Zinecard®, Zoladex®, Zoledronic acid, Zolinza, Zometa®, and Zytiga®.

Asparaginase

Asparaginase, an antileukemic enzyme that degrades the nonessential amino acid asparagine is a chemotherapy drug most commonly used to treat acute lymphoblastic leukemia (ALL). It can also be used to treat some other blood disorders. Asparaginase is also known in the art as, e.g., Erwinase, Crisantaspase or L-asparaginase. Asparaginase catalyzes the conversion of L-asparagine to aspartic acid and ammonia, thus depriving the leukemic cell of circulating asparagine, which leads to cell death.

In one embodiment, the asparaginase is L-asparaginase (Elspar), pegaspargase (PEG-asparaginase; Oncaspar), SC-PEG asparaginase (Calaspargase pegol, and Erwinia asparaginase (Erwinaze Recombinant Crisantaspase, or Recombinant Crisantaspase with half-life extension by pegylation or PASylation).

L-asparaginase (Elspar), pegaspargase (PEG-asparaginase; Oncaspar), and SC-PEG asparaginase (Calaspargase pegol) are all based on the Escherichia coli asparaginase gene ansB, either in its native form or conjugated to polyethylene glycol (pegylated), which encodes a gene product having a sequence of SEQ ID NO: 3.

(SEQ ID NO: 3)
MEFFKKTALAALVMGFSGAALALPNITILATGGTIAGGGDSATKSNY
TVGKVGVENLVNAVPQLKDIANVKGEQVVNIGSQDMNDNVWLTLAKK
INTDCDKTDGFVITHGTDTMEETAYFLDLTVKCDKPVVMVGAMRPST
SMSADGPFNLYNAVVTAADKASANRGVLVVMNDTVLDGRDVTKTNTT
DVATFKSVNYGPLGYIHNGKIDYQRTPARKHTSDTPFDVSKLNELPK
VGIVYNYANASDLPAKALVDAGYDGIVSAGVGNGNLYKSVFDTLATA
AKTGTAVVRSSRVPTGATTQDAEVDDAKYGFVASGTLNPQKARVLLQ
LALTQTKDPQQIQQIFNQY

The Erwinia asparaginases (Erwinaze, Recombinant Crisantaspase, or Recombinant Crisantaspase with half-life extension) are based on the ansB gene from Erwinia chrysanthemi (also known as Dickeya chrysanthemi), either in its native form, conjugated to polyethylene glycol (pegylated), or conjugated to a flexible repetitive hydrophilic sequence of proline, alanine, and serine amino acids (PASylation), which encodes a gene product having a sequence of SEQ ID NO: 4.

(SEQ ID NO: 4)
MERWFKSLFVLVLFFVFTASAADKLPNIVILATGGTIAGSAATGTQT
TGYKAGALGVDTLINAVPEVKKLANVKGEQFSNMASENMTGDVVLKL
SQRVNELLARDDVDGVVITHGTDTVEESAYFLHLTVKSDKPVVFVAA
MRPATAISADGPMNLLEAVRVAGDKQSRGRGVMVVLNDRIGSARYIT
KTNASTLDTFKANEEGYLGVIIGNRIYYQNRIDKLHTTRSVFDVRGL
TSLPKVDILYGYQDDPEYLYDAAIQHGVKGIVYAGMGAGSVSVRGIA
GMRKAMEKGVVVIRSTRTGNGIVPPDEELPGLVSDSLNPAHARILLM
LALTRTSDPKVIQEYFHTY

In one embodiment, the asparaginase encodes a gene product having a sequence that comprises a sequence with at least 80%, at least 85%, at least 90%, at least 95%, at least 96%, at least 97%, at least 98%, at least 99% or greater sequence identity to the sequence of SEQ ID NO: 23 or 24. In one embodiment, the asparaginase encodes a gene product having a sequence that comprises the entire sequence of SEQ ID NO: 3 or 4. In another embodiment, the asparaginase encodes a gene product having a sequence of SEQ ID NO: 3 or 4, wherein the fragment retains the desired function of asparaginase, e.g., the antileukemic enzymatic activity.

Methods for purifying and delivering asparaginases, and compositions comprising asparaginases are further described in, e.g., U.S. Pat. Nos. 3,440,142; 3,511,754; 3,511,755; 3,597,323; 3,652,402; 3,620,925; 3,686,072; 3,773,624; 4,617,271; 6,368,845; 7,666,652; 9,181,552; 9,920,311; 10273444; U.S. Patent Publication No. 2002/0102251; 2003/0186380; 2010/00183765; 2012/0100249; 2013/0023029; and international Application No. WO1999/039732; the contents of which are incorporated herein by reference in their entireties.

Agents that Inhibit G6PD

In one aspect, an agent that inhibits G6PD is administered in combination with an asparaginase to a subject having cancer, e.g., colorectal cancer. In one embodiment, the agent that inhibits G6PD is a small molecule, an antibody or antibody fragment, a peptide, an antisense oligonucleotide, a genome editing system, or an RNAi.

An agent described herein targets G6PD for its inhibition. An agent is considered effective for inhibiting G6PD if, for example, upon administration, it inhibits the presence, amount, activity and/or level of G6PD in the cell.

An agent can inhibit e.g., the transcription, or the translation of G6PD in the cell (e.g., G6PD expression). An agent can inhibit the activity or alter the activity (e.g., such that the activity no longer occurs, or occurs at a reduced rate) of G6PD in the cell (e.g., inhibits the enzymatic activity of G6PD).

In one embodiment, an agent that inhibits G6PD promotes programmed cell death, e.g., kills the cell. To determine is an agent is effective at inhibiting G6PD, mRNA and protein levels of a given target (e.g., G6PD) can be assessed using RT-PCR and western-blotting, respectively. Biological assays that detect the activity of G6PD can be used to assess if programmed cell death has occurred. In other embodiments, the biological assay is selected from the group consisting of fluorescent spot test, spectrophotometric assay, enzyme activity assay, and cytochemical assay.

In one embodiment, an agent that inhibits the level and/or activity of G6PD by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the level and/or activity of G6PD prior to administration of the agent, or the level and/or activity of G6PD in a population of cells that was not in contact with the agent. Inhibition of G6PD will induce asparaginase sensitivity.

The agent may function directly in the form in which it is administered. Alternatively, the agent can be modified or utilized intracellularly to produce something which inhibits G6PD, such as introduction of a nucleic acid sequence into the cell and its transcription resulting in the production of the nucleic acid and/or protein inhibitor of G6PD. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In one embodiment, the agent that inhibits G6PD is conjugated to an E3 ubiquitin ligase recruitment element. As used herein, “conjugated” refers to two or more smaller entities (e.g., an agent and a E3 ubiquitin ligase recruitment element) that are linked, connected, associated, bonded (covalently or non-covalently), or any combination thereof, to form a larger entity. The conjugated E3 ubiquitin ligase recruitment element recruits an E3, which mediates the transfer of an ubiquitin from an E2 to the protein substrate. Binding of an ubiquitin to a protein substrate marks the protein for degradation via the ubiquitin proteasome system. Thus, an inhibitor of G6PD conjugated to an E3 ubiquitin ligase recruitment element would, e.g., bind to G6PD and subsequently promote its degradation. E3 ubiquitin ligase recruitment elements can include, but are not limited to, thalidomide, lenalidomide, pomalidomide, or a VHL ligand that mimics the hydroxyproline degradation motif of HIF1-alpha. Chemical structures for exemplary E3 ubiquitin ligase recruitment element are presented herein in Table 3, and are further described in, e.g., Pavia, S L, and Crews, C M. Current Opinion in Chemical Biology. 2019. 50; 111-119, the contents of which are incorporated herein by reference in its entirety. Use of conjugated E3 ubiquitin ligase recruitment elements are further described in U.S. Pat. Nos. 7,208,157B2 and 9,770,512, the contents of which are incorporated herein by reference in its entirety.

In one embodiment, an agent conjugated to an E3 ubiquitin ligase recruitment element further comprises a linker. It is specifically contemplated herein that the specifications of the linker (e.g., length, sequence, etc.) would be optimized for greatest efficacy of the small molecule and E3 ubiquitin ligase recruitment element. For example, a linker would be designed such that it does not interfere with binding of the agent to its target (e.g., the binding pocket on the protein of interest) or the transfer of the ubiquitin from the E2 to the protein substrate.

In various embodiments, the agent that inhibits G6PD is an antibody or antigen-binding fragment thereof, or an antibody reagent that is specific for G6PD. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the antibody or antibody reagent binds to an amino acid sequence that corresponds to the amino acid sequence encoding G6PD (SEQ ID NO: 2).

In another embodiment, the anti-G6PD antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-G6PD antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., G6PD, and inhibit G6PD activity and/or expression.

In one embodiment, an anti-G6PD antibody or antibody reagent is conjugated to an E3 ubiquitin ligase recruitment element. In one embodiment, the anti-G6PD antibody or antibody reagent conjugated to an E3 ubiquitin ligase recruitment element further comprises a linker.

In one embodiment, the agent that inhibits G6PD is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., G6PD. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits G6PD may comprise at least 5, at least 10, at least 15, at least 20, at least 25, at least 30, or more bases complementary to a portion of the coding sequence of the human G6PD gene (e.g., SEQ ID NO: 1), respectively.

In one embodiment, G6PD is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits G6PD by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. G6PD. In some embodiments of any of the aspects, the agent is siRNA that inhibits G6PD. In some embodiments of any of the aspects, the agent is shRNA that inhibits G6PD.

One skilled in the art would be able to design siRNA, shRNA, or miRNA to target G6PD, e.g., using publically available design tools. siRNA, shRNA, or miRNA is commonly made using companies such as Dharmacon (Layfayette, CO) or Sigma Aldrich (St. Louis, MO).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

In one embodiment, the agent is miRNA that inhibits G6PD. MicroRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of the target gene (e.g., G6PD), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., G6PD, for its downregulation, for example by transfecting the siRNA, shRNA, or miRNA into cells and detecting the levels of a gene (e.g., G6PD) found within the cell via western-blotting.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., an G6PD inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

Administration

In some embodiments, the methods described herein relate to treating a subject having or diagnosed as having cancer (e.g., colorectal cancer or leukemia) comprising administering an agent that inhibits G6PD in combination with an asparaginase as described herein. In some embodiments, the methods described herein relate to treating a subject having or diagnosed as having cancer comprising a mutation that results in G6PD deficiency comprising administering an asparaginase as described herein.

Subjects having cancer can be identified by a physician using current methods of diagnosing a condition. Symptoms and/or complications of cancer, which characterize this disease and aid in diagnosis are well known in the art and include but are not limited to, fatigue or extreme tiredness that does not get better with rest; weight loss or gain of 10 pounds or more for no known reason; eating problems such as not feeling hungry; trouble swallowing, belly pain, or nausea and vomiting; swelling or lumps anywhere in the body; thickening or lump in the breast or other part of the body; pain, especially new or with no known reason; that does not go away or gets worse; skin changes such as a lump that bleeds or turns scaly, a new mole or a change in a mole, a sore that does not heal, or a yellowish color to the skin or eyes (e.g., jaundice); cough or hoarseness that does not go away; unusual bleeding or bruising for no known reason; change in bowel habits, such as constipation or diarrhea, that does not go away or a change in how stools appear; bladder changes such as pain when passing urine, blood in the urine or needing to pass urine more or less often; fever or nights sweats; headaches; vision or hearing problems; mouth changes such as sores, bleeding, pain, or numbness.

Symptoms and/or complications of colorectal cancer which characterize these conditions and aid in diagnosis are well known in the art and include but are not limited to, a persistent change in bowel habits, including diarrhea or constipation or a change in the consistency of stool; rectal bleeding or blood in stool; persistent abdominal discomfort, such as cramps, gas or pain; a feeling that the bowel doesn't empty completely; weakness or fatigue; or unexplained weight loss.

Tests that may aid in a diagnosis of, e.g., cancer, include blood tests and non-invasive imaging. A family history of a particular cancer will also aid in determining if a subject is likely to have the condition or in making a diagnosis of cancer. Tests that may aid in a diagnosis of, e.g. colorectal cancer include, but are not limited to, colonoscopy, proctoscopy, colon or rectum biopsy, stool tests (e.g., Cologuard®), genetic testing (e.g., for changes in the KRAS, NRAS, or BRAF genes; microsatellite instability (MSI); changes in any of the mismatch repair (MMR) genes (MLH1, MSH2, MSH6, and PMS2); changes in the EPCAMgene), CT-guided needle biopsy, ultrasound, MRI, PET scan, or exposure to risk factors for colorectal cancer (e.g. lack of regular physical activity; a diet low in fruit and vegetables; a low-fiber and high-fat diet, or a diet high in processed meats; overweight and obesity; alcohol consumption; or tobacco use) can also aid in determining if a subject is likely to have colorectal cancer or in making a diagnosis of colorectal cancer.

The agents described herein (e.g., an agent that inhibits G6PD) and an asparaginase can be administered in combination to a subject having or diagnosed as having cancer (e.g., colorectal cancer or leukemia). Administration of an agent or asparaginase described herein can be performed in a variety of manners, for example, in a single dose, in reoccurring multiple doses, via continuous infusion, via pulsed administration. In one embodiment, an agent or asparaginase described herein can be administered to a subject at least once every 1, 2, 3, 4, 5, 6, 7, 8, 9, 10, 11, 12, 13, 14, 15, 16, 17, 18, 19, 20, 21, 22, 23, or 24 hours; or every 1, 2, 3, 4, 5, 6, or 7 days; or every 1, 2, 3, or 4 weeks; or every 1, 2, 3, 4, 5, 6, 7, 8, 9, 10, 11, or 12 months, or more. It is specifically contemplated herein that the dosing of an agent or asparaginase described herein is determined based on the half-life of the agent, e.g., such that the effect of the agent (for example, inhibition of G6PD) is continuous, or nearly continuous, in the subject. For example, if the half-life of a given G6PD inhibitor is 12 hours, it would be administered every 12 hours to the subject such that it maintains continuous inhibition of G6PD in the subject.

In one embodiment, the agent that inhibits G6PD and the asparaginase are administered in the same manner, e.g., the agent that inhibits G6PD and the asparaginase are administered in a single dose, in multiple doses, via continuous infusion, via pulsed administration. In one embodiment, the agent that inhibits G6PD and the asparaginase are administered in different manners, e.g., the agent that inhibits G6PD is administered via continuous infusion, and the asparaginase is administered in a single dose.

In some embodiments, the methods described herein comprise administering an effective amount of the agents to a subject in order to alleviate at least one symptom of a given cancer. As used herein, “alleviating at least one symptom of a given cancer” is ameliorating any condition or symptom associated with cancer. As compared with an equivalent untreated control, such reduction is by at least 5%, 10%, 20%, 40%, 50%, 60%, 80%, 90%, 95%, 99% or more as measured by any standard technique. A variety of means for administering the agents and/or an asparaginase described herein to subjects are known to those of skill in the art. In one embodiment, the agent is administered systemically or locally (e.g., to the affected organ, e.g., the colon). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

The term “effective amount” as used herein refers to the amount of an agent (e.g., an agent that inhibits G6PD) and/or an asparaginase that can be administered to a subject having or diagnosed as having cancer (e.g., colorectal cancer or leukemia) needed to alleviate at least one or more symptom of cancer. The term “therapeutically effective amount” therefore refers to an amount of an agent and/or an asparaginase that is sufficient to provide a particular anti-cancer effect when administered to a typical subject. An effective amount as used herein, in various contexts, would also include an amount of an agent and/or an asparaginase sufficient to delay the development of a symptom of cancer, alter the course of a symptom of cancer (e.g., slowing the progression of cancer), or reverse a symptom of cancer. Thus, it is not generally practicable to specify an exact “effective amount”. However, for any given case, an appropriate “effective amount” can be determined by one of ordinary skill in the art using only routine experimentation.

In one embodiment, the agent and/or an asparaginase is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent and/or an asparaginase as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

Combination Treatment

In one aspect, the agent and an asparaginase described herein are administered in combination for the treatment of cancer. Administered “in combination,” as used herein, means that two (or more) different treatments (e.g., an asparaginase and an agent that inhibits G6PD are delivered to the subject during the course of the subject's affliction with the disorder, e.g., the two or more treatments are delivered after the subject has been diagnosed with the disorder (e.g., cancer) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent and/or an asparaginase described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

When administered in combination, the agent and an asparaginase, or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect.

It is specifically contemplated herein that the treatments described herein can be administered in combination with another anti-cancer therapy.

Parenteral Dosage Forms

Parenteral dosage forms of an agents described herein and/or an asparaginase can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Controlled and Delayed Release Dosage Forms

In some embodiments of the aspects described herein, an agent and/or an asparaginase is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

Efficacy

The efficacy of an agents described herein and/or an asparaginase, e.g., for the treatment of cancer, can be determined by the skilled practitioner. However, a treatment is considered “effective treatment,” as the term is used herein, if one or more of the signs or symptoms of cancer are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated (e.g., cancer) according to the methods described herein or any other measurable parameter appropriate. Efficacy can also be measured by a failure of an individual to worsen as assessed by hospitalization, or need for medical interventions (i.e., progression of cancer). Methods of measuring these indicators are known to those of skill in the art and/or are described herein.

Efficacy can be assessed in animal models of a condition described herein, for example, a mouse model or an appropriate animal model of a given cancer, as the case may be. When using an experimental animal model, efficacy of treatment is evidenced when a statistically significant change in a marker is observed, e.g., a reduction in tumor size, or prevention of metastasis.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as described in any one of the following numbered paragraphs:

1. A method for treating cancer, the method comprising: administering to a subject having cancer an asparaginase and an agent that inhibits glucose 6 phosphate dehydrogenase (G6PD).

2. The methods of any of the above paragraphs, wherein the cancer is selected from the list consisting of: a carcinoma, a melanoma, a sarcoma, a myeloma, a leukemia, and a lymphoma.

3. The method of any of the above paragraphs, wherein the cancer is a solid tumor.

4. The method of any of the above paragraphs, wherein the cancer is colon cancer.

5. The method of any of the above paragraphs, wherein the leukemia is acute myeloid leukemia (AML), Chronic myeloid leukemia (CML), Acute lymphocytic leukemia (ALL), and Chronic lymphocytic leukemia (CLL).

6. The method of any of the above paragraphs, wherein the cancer is resistant to an asparaginase.

7, The method of any of the above paragraphs, wherein the cancer is not resistant to an asparaginase.

8. The method of any of the above paragraphs, wherein the asparaginase is selected from the group consisting of: L-asparaginase (Elspar), pegaspargase (PEG-asparaginase; Oncaspar), SC-PEG asparaginase (Calaspargase pegol, and Erwinia asparaginase (Erwinaze).

9. The method of any of the above paragraphs, wherein the agent that inhibits G6PD is selected from the group consisting of a small molecule, an antibody, a peptide, a genome editing system, an antisense oligonucleotide, and an RNAi.

10. The method of any of the above paragraphs, wherein the RNAi is a microRNA, an siRNA, or a shRNA.

11. The method of any of the above paragraphs, wherein inhibiting G6PD is inhibiting the expression level and/or activity of G6PD.

12. The method of any of the above paragraphs, wherein the expression level and/or activity of G6PD is inhibited by at least 50%, at least 60%, at least 70%, at least 80%, at least 90%, or more as compared to an appropriate control.

13. A method of treating cancer, the method comprising:
a. receiving the results of an assay that identifies a subject as having a deficiency in G6PD.
b. administering an asparaginase to a subject who has been identified having a deficiency in G6PD.

14. The methods of any of the above paragraphs, wherein the cancer is selected from the list consisting of: a carcinoma, a melanoma, a sarcoma, a myeloma, a leukemia, or a lymphoma.

15. The method of any of the above paragraphs, wherein the cancer is a solid tumor.

16. The method of any of the above paragraphs, wherein the cancer is colon cancer.

17. The method of any of the above paragraphs, wherein the cancer is metastatic.

18. The method of any of the above paragraphs, wherein the cancer is resistant to an asparaginase.

19. The method of any of the above paragraphs, wherein the cancer is not resistant to an asparaginase.

20. The method of any of the above paragraphs, wherein the biological sample is a biopsied sample, a tissue sample or a blood sample.

21. The method of any of the above paragraphs, wherein the biopsied sample is a tumor sample.

22. The methods of any of the above paragraphs, wherein the asparaginase is selected from the group consisting of: L-asparaginase (Elspar), pegaspargase (PEG-asparaginase; Oncaspar), SC-PEG asparaginase, Calaspargase pegol (Cal-PEG; SHP663), Erwinia asparaginase (Erwinaze), cristantaspase, and Asparaginase medac.

23. The methods of any of the above paragraphs, wherein the subject has previously been administered an anti-cancer therapy.

24. The methods of any of the above paragraphs, wherein the subject has not previously been administered an anti-cancer therapy.

25. A method for treating cancer, the method comprising:
a. obtaining a biological sample from a subject having cancer;
b. assaying the sample and identifying a deficiency in G6PD
c. administering an asparaginase to a subject who has been identified with G6PD deficiency.

### EXAMPLES

### Example 1

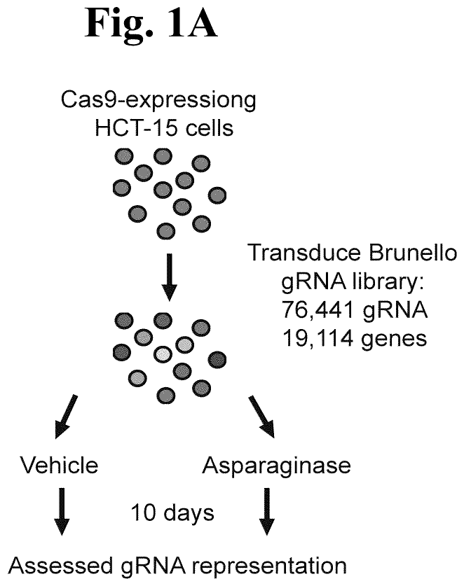

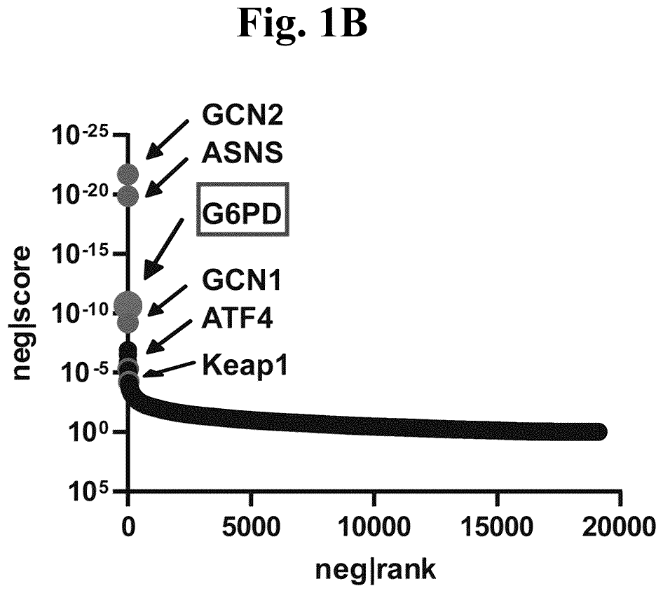

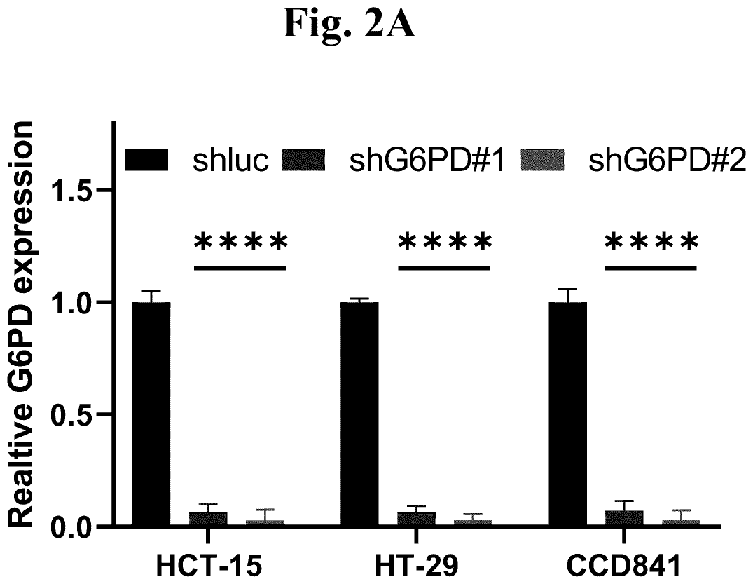

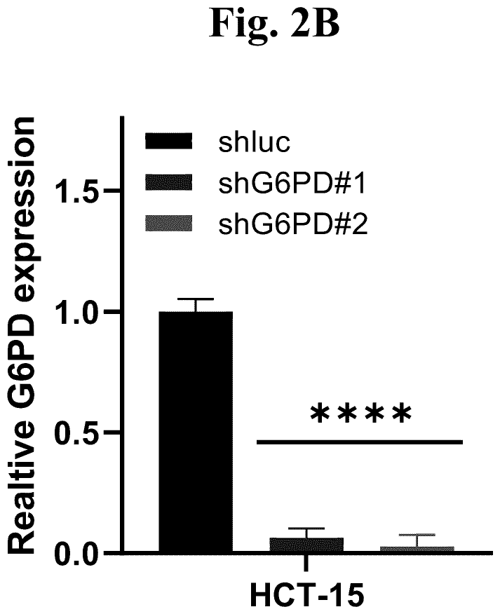

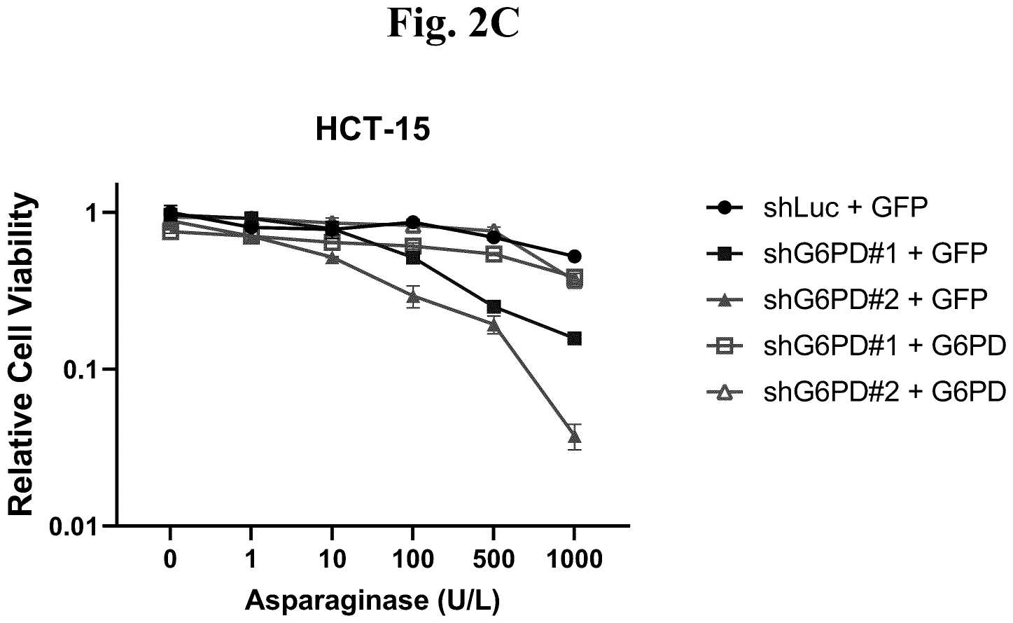

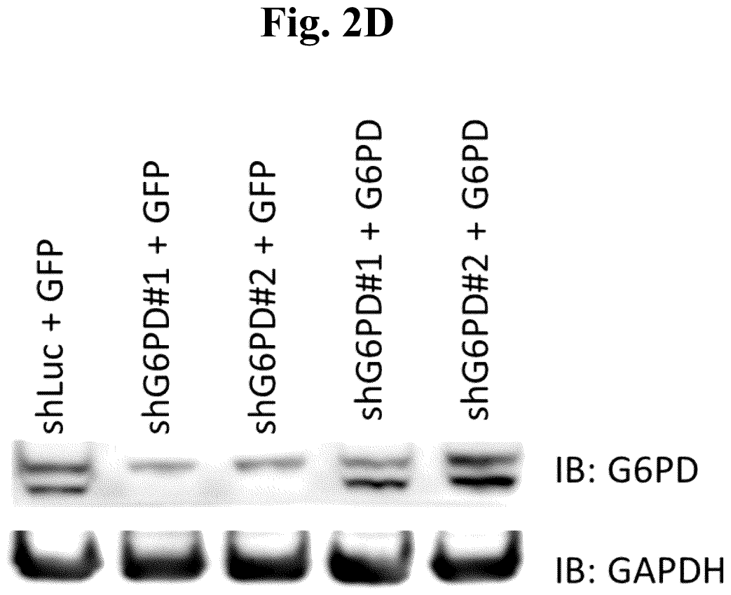

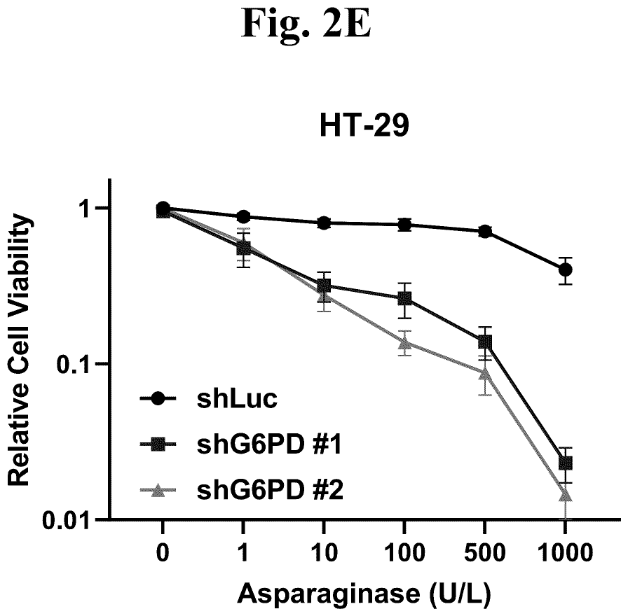

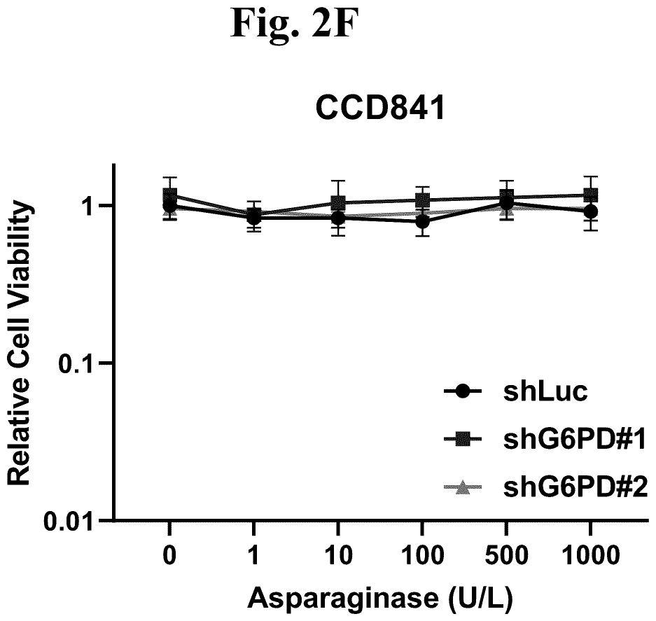

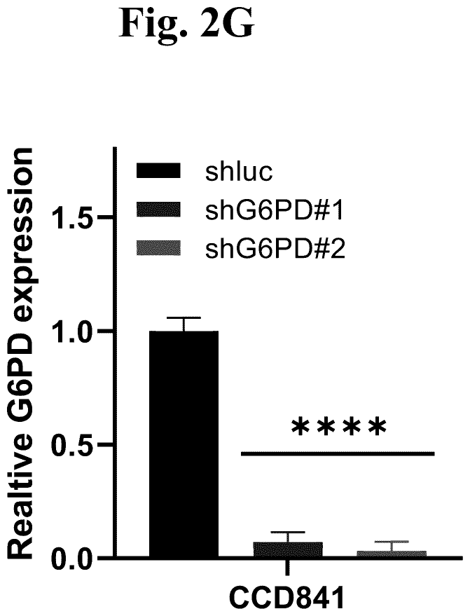

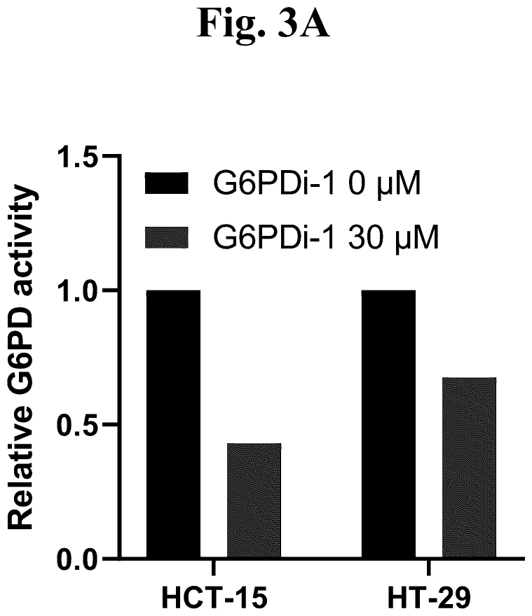

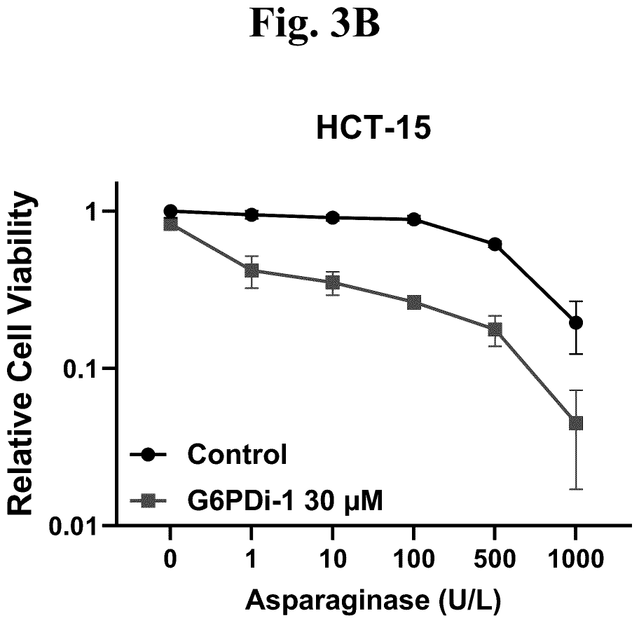

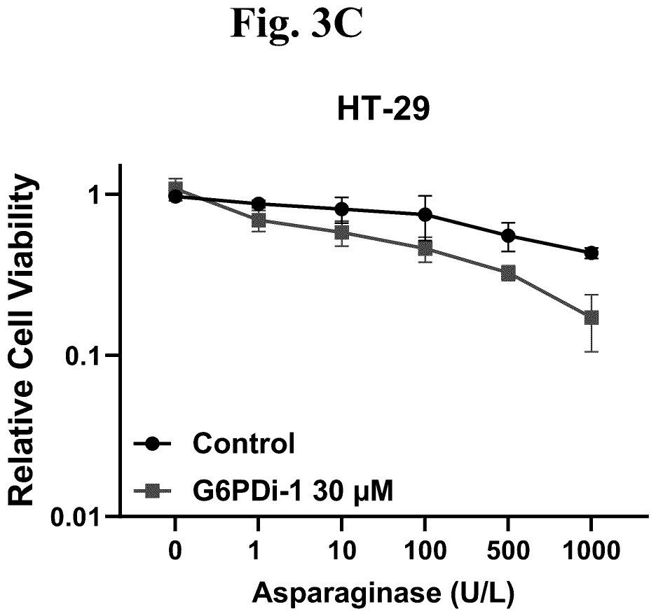

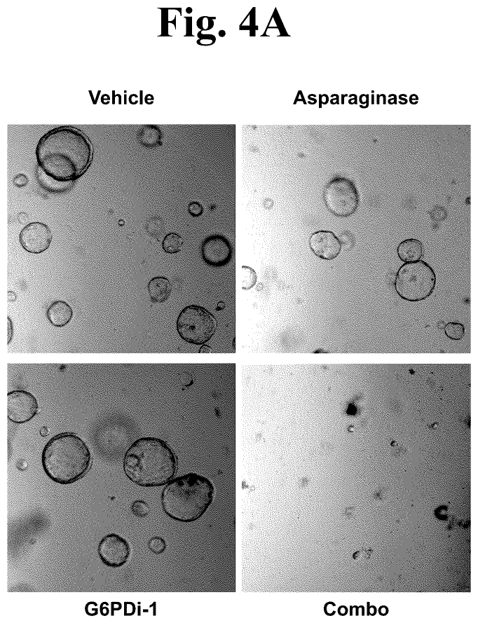

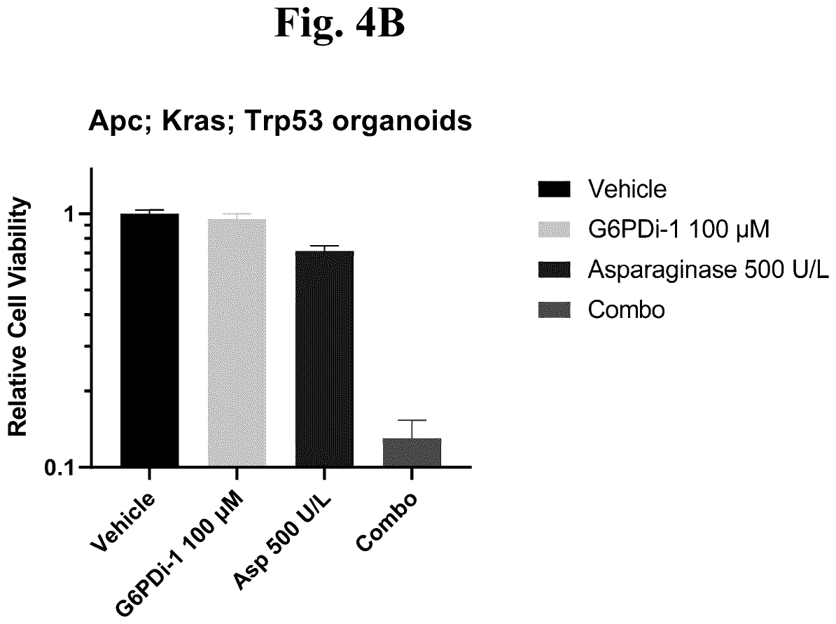

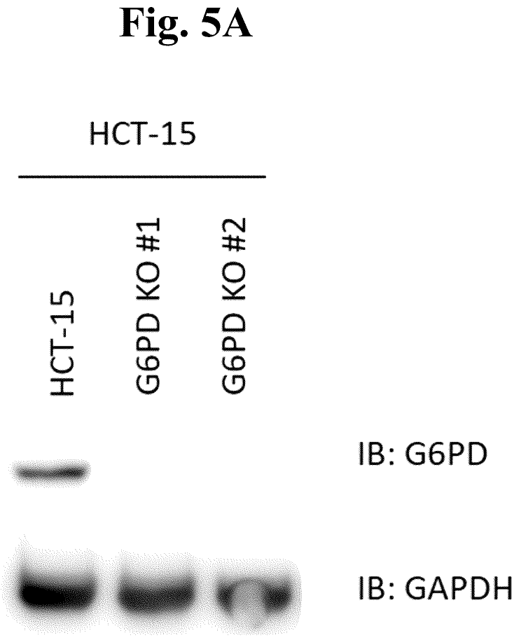

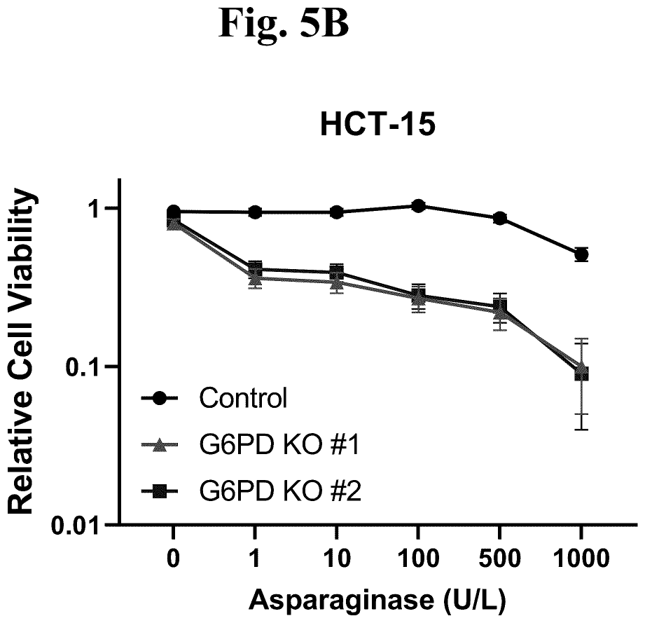

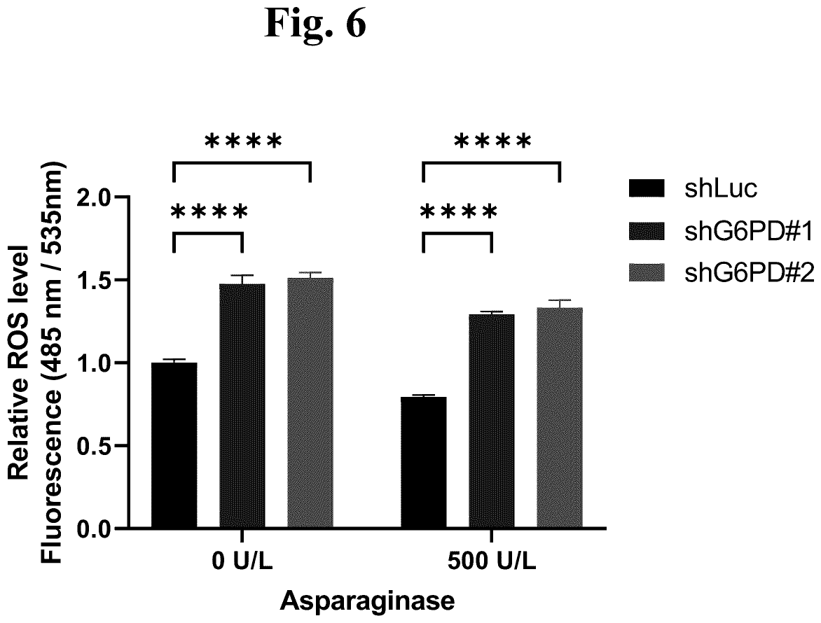

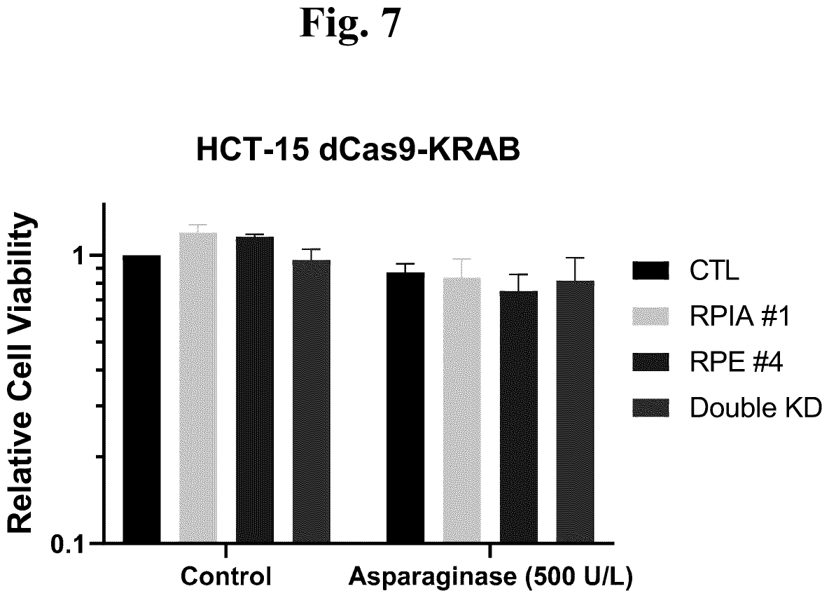

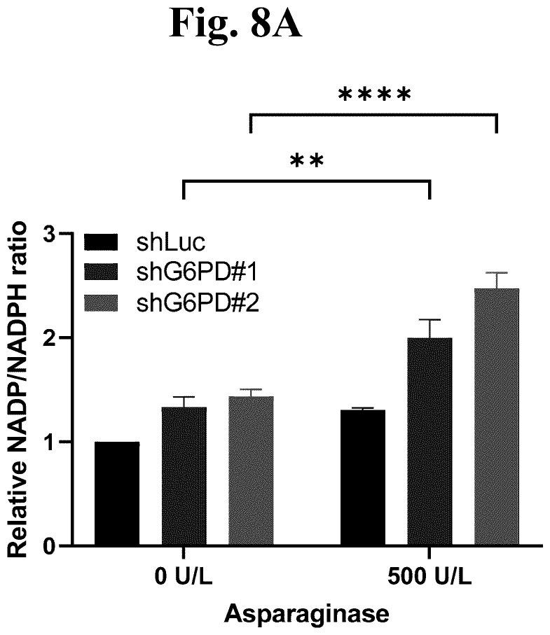

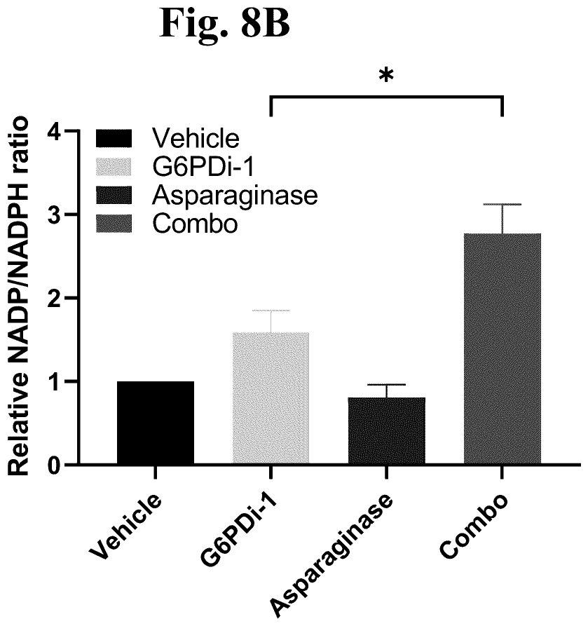

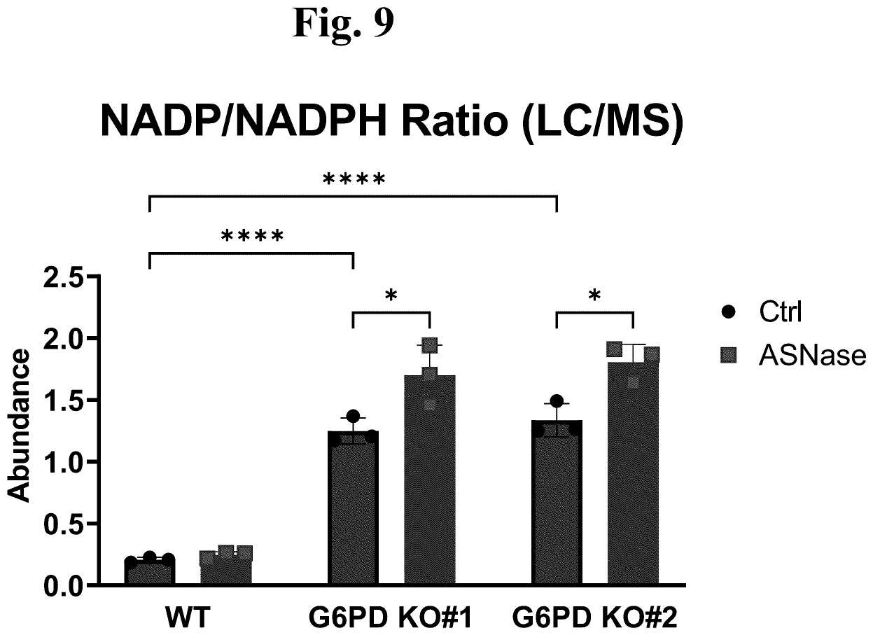

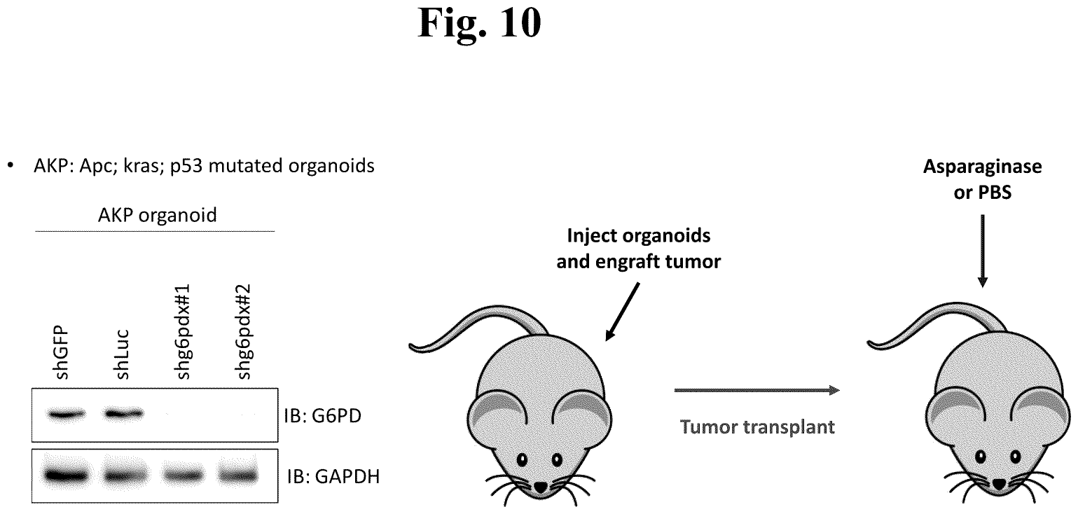

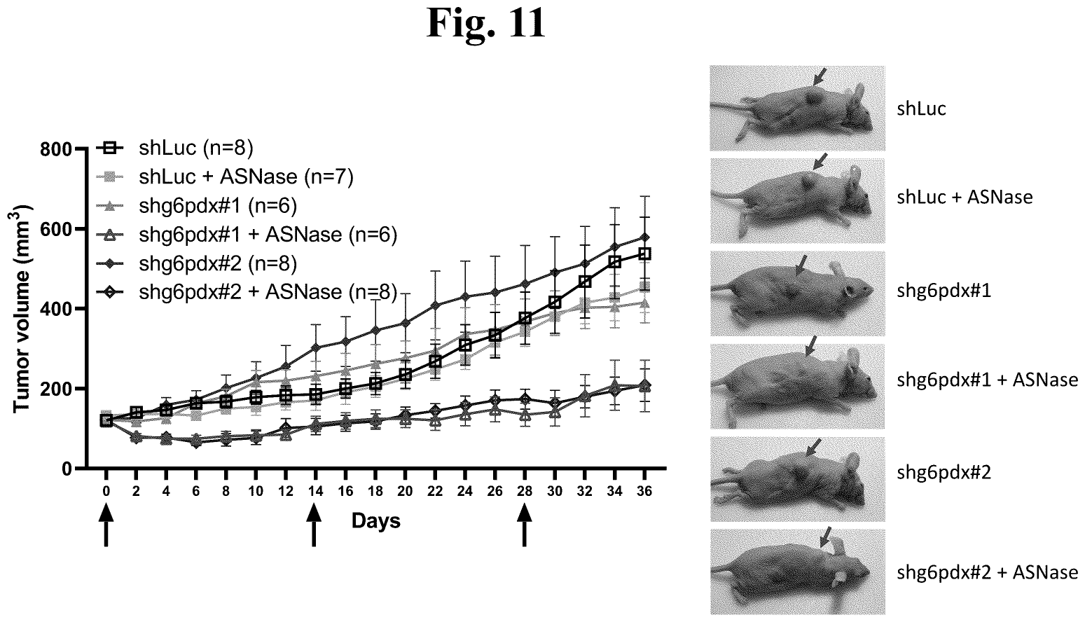

A genome-wide CRISPR screen was conducted to identify new mechanisms to induce asparaginase sensitivity in colon cancer (FIG. 1a). The screen revealed the depletion of select genes results in increase in asparaginase sensitivity, including depleting of G6PD (FIG. 1b). G6PD is a critical enzyme that is required for the pentose phosphate pathway. Knockdown of G6PD expression and activity using short hairpin RNAs (shRNAs) resulted in colorectal cell lines having increased sensitivity to asparaginase (FIGS. 2a-2g) Similarly, a small molecule G6PD inhibitor, G6PDi-1 could also reduce G6PD activity, and subsequently induce sensitivity to asparaginase in colorectal cell lines (FIGS. 3a-3c). Small molecule inhibition of G6PD using G6PDi-1 could also induce asparaginase sensitivity in intestinal organoids that were designed to recapitulate the genetics of human colorectal cancer (FIGS. 4a-4b). Furthermore, G6PD knock-out in cells lines induced asparaginase sensitivity (FIGS. 5a-5b). Next, potential mechanisms for G6PD's mediation of asparaginase response were investigated. Reactive oxygen species (ROS) levels were assessed in HCT cells that were first transduced with shRNA that reduced G6PD expression and then treated with asparaginase (FIG. 6). ROS production is not a major mechanism through which G6PD deficiency induces asparaginase sensitivity. Furthermore, the ribose 5-phosphate pathway was investigated. RPIA and RPE enzyme inhibition did not affect asparaginase sensitivity in GCT-15 cells (FIG. 7). NADPH and NADH production was assessed by measuring NADP/NADPH in HCT-15 cells first transduced with shRNA, or treated with G6PDi-1 to reduce G6PD levels and then treated with asparaginase or a vehicle control. NAPD/NADPH ratio was increased in cells with less G6PD expression, and asparaginase treatment further increased the NADP/NADPH ratio in G6PD depleted cells (FIGS. 8a-8b, and 9). Finally, a G6PDx deficient mouse intestinal organoid model of colorectal cancer was generated by injecting AKP organoids into a mouse to engraft a tumor. These organoids have Apc deficiency, Kras activating mutations, and Tp53 loss, and produce very little G6PD (FIG. 10). This model showed lack of G6PD led to asparaginase sensitization as tumor volumes were reduced following asparaginase treatment in the G6pdx-deficient, but not the control, organoid bodels of colorectal cancer (FIG. 11).

1) A method for treating cancer, the method comprising: administering to a subject having cancer an asparaginase and an agent that inhibits glucose 6 phosphate dehydrogenase (G6PD).

2) The methods of claim 1, wherein the cancer is selected from the list consisting of: a carcinoma, a melanoma, a sarcoma, a myeloma, a leukemia, and a lymphoma.

3) The method of claim 1, wherein the cancer is a solid tumor.

4) The method of claim 1, wherein the cancer is colon cancer.

5) The method of claim 2, wherein the leukemia is acute myeloid leukemia (AML), Chronic myeloid leukemia (CML), Acute lymphocytic leukemia (ALL), and Chronic lymphocytic leukemia (CLL).

6) The method of claim 1, wherein the cancer is resistant to an asparaginase.

7) The method of claim 1, wherein the cancer is not resistant to an asparaginase.

8) The method of claim 1, wherein the asparaginase is selected from the group consisting of: L-asparaginase (Elspar), pegaspargase (PEG-asparaginase; Oncaspar), SC-PEG asparaginase (Calaspargase pegol, and Erwinia asparaginase (Erwinaze).

9) The method of claim 1, wherein the agent that inhibits G6PD is selected from the group consisting of a small molecule, an antibody, a peptide, a genome editing system, an antisense oligonucleotide, and an RNAi.

10) The method of claim 9, wherein the RNAi is a microRNA, an siRNA, or a shRNA.

11) The method of claim 1, wherein inhibiting G6PD is inhibiting the expression level and/or activity of G6PD.

12) The method of claim 11, wherein the expression level and/or activity of G6PD is inhibited by at least 50%, at least 60%, at least 70%, at least 80%, at least 90%, or more as compared to an appropriate control.

13) A method of treating cancer, the method comprising:
a. receiving the results of an assay that identifies a subject as having a deficiency in G6PD.
b. administering an asparaginase to a subject who has been identified having a deficiency in G6PD.

14) The methods of claim 13, wherein the cancer is selected from the list consisting of: a carcinoma, a melanoma, a sarcoma, a myeloma, a leukemia, or a lymphoma.

15) The method of claim 13, wherein the cancer is a solid tumor.

16) The method of claim 13, wherein the cancer is colon cancer.

17) The method of claim 13, wherein the cancer is metastatic.

18) The method of claim 13, wherein the cancer is resistant to an asparaginase.

19) The method of claim 13, wherein the cancer is not resistant to an asparaginase.

20) The method of claim 13, wherein the biological sample is a biopsied sample, a tissue sample or a blood sample.

21) The method of claim 20, wherein the biopsied sample is a tumor sample.

22) The methods of any of claim 13, wherein the asparaginase is selected from the group consisting of: L-asparaginase (Elspar), pegaspargase (PEG-asparaginase; Oncaspar), SC-PEG asparaginase, Calaspargase pegol (Cal-PEG; SHP663), Erwinia asparaginase (Erwinaze), cristantaspase, and Asparaginase medac.

23) The methods of any of claim 13, wherein the subject has previously been administered an anti-cancer therapy.

24) The methods of any of claim 13, wherein the subject has not previously been administered an anti-cancer therapy.

25) A method for treating cancer, the method comprising:
a. obtaining a biological sample from a subject having cancer;
b. assaying the sample and identifying a deficiency in G6PD
c. administering an asparaginase to a subject who has been identified with G6PD deficiency.
